(19) United States
(12) Patent Application Publication
(10) Pub. No.: US 2026/0110680 A1
(43) Pub. Date: Apr. 23, 2026

(54) PROCESS FOR MEASURING THE CARBON REMOVAL POTENTIAL AND DURABILITY OF PYROLYZED CARBON

(71) Applicant: Carba Inc., Edina, MN (US)

(72) Inventor: Andrew JONES, Edina, MN (US)

(73) Assignee: Carba Inc., Edina, MN (US)

(21) Appl. No.: 19/361,700

(22) Filed: Oct. 17, 2025

Related U.S. Application Data
(60) Provisional application No. 63/708,851, filed on Oct. 18, 2024.

(51) Int. Cl. G01N 33/497; C12Q 1/02; G01N 1/44; G01N 33/24

(52) U.S. Cl. CPC G01N 33/4977; C12Q 1/02; G01N 1/44; G01N 33/24

(57) ABSTRACT

Provided is a process for the quantification and determination of the stable fraction of carbon in a sample of pyrolyzed carbon in anoxic storage. The process comprises pyrolyzing an organic material and measuring the carbon content of a sample of pyrolyzed carbon. A sample of the pyrolyzed carbon is sealed in a vial in an oxygen-free atmosphere, and a mixture of nutrients and microbes is added thereto. The temperature of the vial is controlled. The volume and species of gases evolved over time is measured. The fraction of carbon lost is calculated based on the gases evolved, the carbon content and mass relative to a control sample.

### CROSS REFERENCE TO RELATED APPLICATIONS

This application claims priority to U.S. Provisional Application Ser. No. 63/708,851 filed Oct. 18, 2024, the complete disclosure of which is incorporated herein by reference in its entirety.

### FIELD

The present application relates to the removal of carbon dioxide from the atmosphere. The present process provides a more accurate method for quantifying the durability/longevity of said removal.

### BACKGROUND

The continued emissions of gasses into Earth's atmosphere has emerged as an existential crisis in the 21st century. Over a century of combustion of fossil fuels has accumulated excess carbon dioxide raising the composition to 414 ppm in 2021, far above the ˜300 ppm CO₂ composition at the beginning of the 20th century. At the current rate of carbon accumulation in the atmosphere, carbon dioxide concentrations in the atmosphere could produce a 5° C. warming by the end of the century, excluding any mitigation. The resulting climate change has been predicted for a century, with increasing sea levels, more powerful storms, and more intense and frequent droughts and heat waves. Catastrophe is predicted for wildlife, the environment, and human civilization, with increased extinction of species, human migration away from climate-impacted areas, and substantial economic costs imposed by these global changes.

Climate change will have sweeping effects on human society including the economy and financial sector. Climate-related shifts in the physical environment can slow economic growth and increase the likelihood of disruption and reductions in output, employment, and business profitability. Humans and animals will face new survival challenges because of climate change. Storms, heat waves, more frequent and intensive droughts, melting glaciers, rising sea levels and warming oceans can directly harm animals, destroy places they live, and wreak havoc on people's livelihoods and communities. Carbon dioxide in the atmosphere has a major impact in creating such climate change.

Carbon management technologies must be able to capture carbon dioxide from the atmosphere and store it with a level of permanence relevant to: (i) the time scale of climate change, (ii) the quantity of carbon to be removed, and (iii) the economics per tonne of captured carbon. These processes remain in development but can be classified by their general approach to obtaining carbon, the chemical steps for converting it to some final form, and the type of carbonaceous product and associated degree of permanence. The complexity of the carbon accumulation and storage technologies has led to an expansive array of process concepts, and an even more complex combination of words and phrases to classify the process classes.

Individuals and companies can work to reduce their carbon emissions; however, permanent removal of carbon dioxide from the atmosphere is the only way to limit the worst effects of climate change and get back to pre-industrial CO₂ levels. A solution for such removal of CO₂ is needed to aid in reducing the sweeping shifts of climate change.

Furthermore, the protection of pyrolyzed carbon from oxygen and/or sunlight is an emerging field to limit degradation of the carbon, and in some cases, provide environmental co-benefits. The stability of pyrolyzed carbon in anoxic environments, however, is not well understood. Methods for the quantification and longevity of stable carbon in anoxic environments are non-existent. A method for measuring and quantifying the stable fraction and durability of pyrolyzed carbon in anoxic environments would be of great benefit, and would enhance the viability of using pyrolyzed carbon to reduce carbon dioxide in the atmosphere.

### SUMMARY

A process is hereby provided for quantifying the durability of pyrolyzed carbon in anoxic storage chambers. The process is used in conjunction with a method of removing carbon dioxide from the atmosphere by converting biomass or organic residues through a pyrolysis process into charcoal or biochar, which is subsequently contained to limit oxygen exposure. The durability of charcoal is improved by limiting oxygen exposure, however, the present process further provides a method for the accurate determination of the carbon lifetime. The present process comprises quantifying the durability of pyrolyzed carbon in different environments through the incubation and inoculation of pyrolyzed carbon and measurement of evolved gases.

In another embodiment, provided is a complete process of pyrolyzing an organic material to produce a pyrolyzed carbon product, and then determining the carbon lifetime.

### DETAILED DESCRIPTION

Carbon from biomass or organic matter can be heated to break molecular bonds and remove oxygen and hydrogen from carbohydrates and lignin in a process known as pyrolysis. This process removes the food value of the carbon and creates highly aromatic carbon structures. The resulting material can be referred to as charcoal or biochar. This pyrolyzed carbon can be applied to soils to improve agricultural productivity, however, in this environment it is subjected to oxidative degradation processes including aerobic microbial and fungal attack, direct oxidation, and free-radical mediated oxidation routes catalyzed with light. To prevent decay and virtually eliminate the risk of decomposition, pyrolyzed carbon samples can be stored in a way to limit oxygen and/or light. This can include placing the pyrolyzed carbon in specially designed vaults above or below ground, pits or mines with the appropriate soil/clay/sand or water cover, and/or landfills. Current methods do not exist to be able to estimate the lifetime and degradation potential of pyrolyzed carbon in these storage environments deprived of oxygen and light.

The present process subjects pyrolyzed organic material or biomass (i.e., charcoal or biochar) to conditions to simulate and accelerate degradation for the purposes of measuring the carbon removal potential over long periods of time in anoxic (oxygen-free) environments. The goal of this process is to determine the duration of stability of fractions of pyrolyzed carbon quickly and efficiently, thus allowing permanence assessments of the total carbon content for downstream carbon crediting.

In one embodiment of the present process a sample of pyrolyzed biomass is placed in a sealed vial and purged of oxygen with an inert gas. In one embodiment the purge gas is nitrogen. In another embodiment the purge gas is a mixture of 20% carbon dioxide with the balance nitrogen. An inoculum is added with nutrients and the mixture, and is held at an elevated temperature for a period of time. The nutrients support replication of microbes in the inoculum. In one embodiment, the temperature is at least 37° C. In another embodiment, the temperature is 55° C. A temperature between 37° C. and 55° C. can generally be employed.

The evolved gases are measured over time (volume and concentration). The incubation is followed until degradation rates become negligible. The amount of gas produced is used to calculate the amount of carbon lost from the pyrolyzed carbon over a given amount of time (usually months). Depending on the conditions and inoculum used, one can determine the fraction of carbon that can be degraded over long periods of time (>1000 years) in anoxic environments.

In one embodiment, the inoculum is comprised of a landfill leachate sample with an active population of anaerobic microbes and methanogens. The inoculum is maintained under specific conditions to maintain microbial growth before inoculation.

In another embodiment, the inoculum is comprised of a sample from an active coal or graphite mine.

In one embodiment, a biochemical methane potential test can be used. The testing provides a measure of methane yield that can be achieved from a given amount of sample under ideal conditions. There are three distinct phases to the procedure. The first step consists of maintaining a culture that will be used to inoculate assays. Culture maintenance requires preparing media and transferring the culture into this media regularly, e.g., every two weeks.

The second step involves initiating the assay. Initiating includes weighing a sample into serum bottles, preparing pyrolyzed carbon media, transferring the media into serum bottles, and inoculating the serum bottles.

The final step of the procedure is to measure methane production from the serum bottles. This step includes measuring both gas volume and gas composition using a gas chromatograph.

In one embodiment, a mixed culture or consortium that is acclimated to growth on pyrolyzed carbon in anerobic conditions is maintained in the laboratory to serve as an inoculum for the tests. This culture must be transferred every 2 weeks or so to maintain the culture in an active state. In addition, the culture should be transferred two weeks prior to use as an inoculum for a test. This is to minimize background methane production associated with the inoculum. A medium which can be used for culture maintenance is described below, as one example.

TABLE 1

| | Amount to add per | |
|---|---|---|
| Component | liter of Media | Unit |
| PO₄ solution | 100 | mL |
| Salt solution | 100 | mL |
| Trace Mineral Solution | 10 | mL |
| Vitamin Solution | 10 | mL |
| Hemin (0.01%) | 10 | mL |
| Resazurin (0.1%) | 2 | mL |
| Deionized Water | 758 | mL |

In one embodiment, the mixed culture or inoculum is combined with a small amount of pyrolyzed carbon. The combination can occur in any suitable vessel from which evolved gases can be measured. For example, a bottle, flask, or test tube, etc.

The volume of evolved gases are then measured, as well as the species of gases. The fraction of carbon lost can then be calculated from the volume of evolved gases. The species of gases would also be helpful. One can then determine the faction of carbon that will be degraded over long periods of time in an anoxic environment. This information can be used for predictions or assertions about carbon permanence which are necessary for determining the value of carbon credits.

In one example, 2 g of pyrolyzed carbon is added to a 125 mL serum bottle with an isobutylene rubber cap. The oxygen is purged from the vial and 94 mL of a media solution are added (Table 1). Following this, 15 mL of an active inoculum are added while maintaining an oxygen free environment. The serum bottles are sealed and allowed to incubate over time at a set temperature, e.g., 37° C. The inoculum is fresh or adequately maintained following best practices. The amount and type of gas is measured using volumetric flowmeters or syringes, and a gas chromatograph with thermal conductivity detector, or other suitable detectors.

In another embodiment, the testing protocol is amended to measure gas production in real time using flow meters and compositional detectors. In another embodiment, the nutrient mixture contains municipal solid waste, derivatives thereof, or synthetic variations. In another embodiment, the nutrient mixture contains a mixture of organic food. Real time data and variable nutrient mixtures can be useful in determining value of carbon credits contingent on evolved carbon assessed in this testing.

Pyrolyzed carbons can also be amended/modified to improve their properties in anoxic environments based on the results of the quantification. Inoculation of the pyrolyzed carbon with methane oxidizing bacteria, and/or nutrients to promote methane oxidizing bacteria can improve the methane consuming capacity and rate of the carbon. In one embodiment, the carbon is mixed with landfill leachate to inoculate and increase the presence of bacteria. In another embodiment, the carbon is mixed with a slurry of waste. In one embodiment the inoculated carbon is allowed to mix in a stirred vessel at an elevated temperature of 25-70° C. or ideally 37-55° C. Nutrients and/or methane/CO₂ may be added to stimulate bacteria growth. Simulating anoxic environmental conditions is also useful for carbon crediting purposes depending on the end-use case of the charcoal or biochar being tested.

In one embodiment, the present process of quantification is used in conjunction with a process of pyrolyzing a biomass to create the carbon product. The biomass can comprise any biomass material that can be pyrolyzed to charcoal product. Often the biomass material comprises a lignocellulose material, e.g., plants, wood, etc.

The process for pyrolyzing carbon can be any suitable process which will provide a pyrolyzed or torrefied carbon product to be placed underground or in a mine under anoxic storage conditions. Torrefaction is a preferred method of creating the pyrolyzed carbon, which is then tested by the present process.

The most carbon-efficient process for converting biomass to a stable solid form is low-temperature pyrolysis, referred to as torrefaction. Lower temperatures minimize cracking reactions that release volatile organic compounds (VOCs) and reduce solid yields. During torrefaction, biomass loses oxygen and hydrogen, producing a carbon-rich solid product with increased heating value. The torrefied material decreases in moisture and takes on a more hydrophobic microstructure, with a compositional change eliminating the fibrous nature of biomass for a more grindable char. The lignocellulosic material transforms from a white-brown-grey virgin material to a dark-grey/black char, with concomitant increase in the degree of carbon-carbon bond unsaturation and aromaticity. These physical and chemical changes to biomass significantly reduce the capability of fungi and bacteria to degrade biomass to volatile products, yielding a stable solid that can sequester carbon long term.

Torrefaction of biomass occurs within a heated reactor chamber generally devoid of molecular oxygen in less than an hour of total reaction time. The control of heat transfer into the biomass is a critical characteristic of reactor design, as the temperature of reacting biomass determines the yield of solid carbon product. Initial heating primarily evaporates water at lower temperatures in the absence of biopolymer degradation; after the drying phase biomass particles further heat until the onset of biopolymer thermolysis. A critical transition temperature of thermally-decomposing cellulose has been identified as 467° C.; above that temperature cellulose rapidly fractures and depolymerizes to volatile products, while lower temperatures lead to faster dehydration rates and higher solid yields. To maximize total productivity of solid char product, torrefaction reactors are designed with many geometries and mechanisms of biomass flow to rapidly heat biomass particles to temperatures below 450° C. Higher biochar yields and avoidance of hydrocarbon cracking are important in maximizing the amount of CO₂ sequestered and thus carbon credits that can be produced with the system.

While any appropriate reactor to affect the desired pyrolysis can be used, a particular example of a preferred reactor is provided herewith. This example is not intended to be limiting. Thus, in one embodiment, the process for converting biomass to a solid carbon product for burial comprises passing the biomass waste into a reactor. The biomass waste is passed along the length of the reactor with heating and mixing. The reactor is heated to a temperature low enough to avoid cracking of the hydrocarbons in the biomass waste, e.g., 300-450° C., 350-450° C., or more preferably 350-400° C. In one embodiment, a twin screw conveyer is used to pass the biomass waste along the length of the reactor. The twin screw conveyor provides the mixing and conveyance along the length of the reactor. A solid carbon product is then collected from the reactor. In another embodiment the reactor is heated between 300-100° C., e.g., 450-1000° C. Higher temperatures promote cracking reactions that lead to higher yields of H₂, light gases and oils.

The reactor can employ a twin screw conveyor to keep the biomass mixed thoroughly while heating, to maintain a uniform temperature profile and faster heat transfer.

Once the carbon product is prepared, the present process of quantification can be processed on the pyrolyzed carbon product as discussed above. The quantification of the carbon product is necessary for calculating the amount of carbon credits that are possible to provide from the system (value of the credits based on permanence is discussed above).

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A process for the quantification and determination of the stable fraction of carbon in a sample of pyrolyzed carbon in anoxic storage, comprising:
pyrolysis of an organic material;
measuring the carbon content of a sample of pyrolyzed carbon;
sealing a sample of pyrolyzed carbon in a vial in an oxygen-free atmosphere;
adding a mixture of nutrients and microbes;
controlling the temperature of the vial;
measuring the volume of gases evolved over time; and
calculating the fraction of carbon lost from the gases evolved, the carbon content and mass relative to a control sample.

2. The process of claim 1, wherein the carbon lost is used to determine the fraction of unstable carbon.

3. The process of claim 2, wherein the unstable carbon is used to reduce the amount of carbon dioxide removal for carbon crediting purposes.

4. The process of claim 1, wherein the nutrients provide ideal conditions for the microbes to replicate.

5. The process of claim 1, wherein the microbes are similar to those found in the anoxic storage conditions.

6. The process of claim 1, wherein the microbes are derived from a landfill sample.

7. The process of claim 1, wherein the microbes are derived from a mine sample or prepared inoculum.

8. The process of claim 1, wherein the volume and species of gases evolved over time are measured.

9. The process of claim 1, wherein the pyrolyzed carbon sample is prepared by subjecting biomass to a pyrolysis process and collecting the pyrolyzed carbon.

10. The process of claim 9, wherein the pyrolysis process comprises:
passing the biomass batchwise into a reactor;
heating the reactor at a temperature of 300-450° C. to avoid cracking of the hydrocarbons in the biomass waste;
passing the biomass waste with heating and mixing along the length of the reactor; and
collecting a solid carbon product from the reactor.

11. The process of claim 10, wherein the reactor comprises a twin screw conveyer.

12. The process of claim 10, wherein the reactor has a secondary volume of heat transfer fluid around the reaction vessel.

13. The process of claim 12, wherein the heat transfer fluid comprises a solar salt composition.

14. The process of claim 11, wherein the twin screw of the twin screw conveyor comprises cuts and folds.

15. The process of claim 11, wherein heat transfer fluid is passed internally through the twin screws of the twin screw conveyor.

16. The process of claim 11, wherein the twin screw conveyor comprises augers with a right hand cut and folded flighting.

17. The process of claim 16, wherein the folded portion of the flighting acts as fingers that lift material as it is conveyed.

18. The process of claim 17, wherein the material is conveyed from 6 o'clock on a flight face to above 2 o'clock.

19. A process comprising inoculating pyrolyzed carbon to increase its methane oxidizing potential.

20. The process of claim 19, wherein landfill leachate is used to inoculate the pyrolyzed carbon.

21. The process of claim 20, wherein the pyrolyzed carbon/inoculate is stirred in a slurry reactor.

22. The process of claim 1, wherein the calculated fraction of carbon lost from the gases evolved is used for determining a value of carbon credits.

23. The process of claim 10, wherein avoiding cracking of the hydrocarbons in the biomass waste maximizes the amount of CO₂ sequestered and carbon credits that can be produced.
